(19) United States
(12) Plant Patent Application Publication
(10) Pub. No.: US 2009/0217432 P1
(43) Pub. Date: Aug. 27, 2009

(54) Catena malbec 'Clone 16'

(50) ;

(76) Inventor: Nicolas Catena, Mendoza (AR)

Correspondence Address: MORRISON & FOERSTER LLP, 425 MARKET STREET, SAN FRANCISCO, CA, 94105-2482, US

(21) Appl. No.: 12/072,553

(22) Filed: Feb. 26, 2008

(51) Int. Cl. A01H 5/00

(52) U.S. Cl. PLT/205

(57) ABSTRACT

A special Malbec grapevine clone from Mendoza Argentina, herewith denominated Catena Malbec ‘Clone 16’ which shows a unique vineyard and winemaking profile from the rest of the Malbec grapevine population in Mendoza, Argentina. This Malbec grapevine clone is characterized by its small cluster size and medium cluster weight; very compact cluster form; very small berry size and weight; medium vigor; medium to high level of millendrage (shot berries); very high level of polyphenol and tannin; very high aromatic intensity and mid palate flavor depth.

### BOTANICAL/COMMERCIAL CLASSIFICATION

Vitis vinifera L.

### VARIETY DENOMINATION

Catena Malbec ‘Clone 16’.

### BACKGROUND OF THE INVENTION

Once known as a variety of Bordeux, the Malbec grape is now being cultivated in South America, including Argentina. Malbec grapes produced delicious wines.

Historically, Argentine vintners did not engage in selecting clones. A less than rigorous attention to clonal selection meant that Malbec vineyards in Mendoza consisted of massal populations, a highly heterogeneous, haphazard mix of clones throughout the vineyard. There is a need for Malbec clones with improved quality based characteristics such as low yield, plant balance, and fruit concentration.

### BRIEF SUMMARY OF THE INVENTION

The present invention relates to a newly selected and distinct clone of the Malbec grapevine, Vitis vinifera L., which will hereinafter be denominated as the Catena Malbec ‘Clone 16’. ‘Clone 16’ has low cluster size and medium cluster weight; very compact cluster form; very small berry size and weight; medium vigor; medium to high level of millendrage (shot berries); very high level of polyphenol and tannin; very high aromatic intensity and mid palate flavor depth.

### BRIEF DESCRIPTION OF THE DRAWINGS

FIG. 1: Total millendrage of overall Malbec grapevine massal population and selected clones.

FIG. 2: Total polyphenols of overall Malbec grapevine massal population and selected clones.

FIG. 3: Total tannins of overall Malbec grapevine massal population and selected clones.

FIG. 4: Aroma and flavour profile of overall Malbec grapevine massal population and selected clones.

FIG. 5: Photograph showing Catena Malbec ‘Clone 16’.

### DETAILED DESCRIPTION OF THE INVENTION

The Malbec grapevine clone of the present invention originated from a very demanding clonal selection carried out in Mendoza, Argentina. The clonal selection process began in 1992 and was conducted solely by Bodegas Esmeralda S.A.

The process began with an identification of different clones of the Malbec grapevine. During three growing seasons, all plants from Lot 18 of Bodegas Esmeralda's Angelica Vineyard, located in the Lunlunta district of the Maipu region of Mendoza, were systematically observed.

The goal was to identify a wide base of genetic profiles. The first criteria were to select those Malbec vines which showed overall good health and good fruit set. The next criterion was diversity, identifying those plants with varying levels of vigor; different dates of budbreak and harvest; and varying sizes of clusters and berries. Malbec vines fitting these criteria were marked for further study.

This preliminary selection process resulted in the identification of 108 different Malbec clonal plants from Lot 18 of Bodegas Esmeralda's Angelica Vineyard, located in the Lunlunta district of the Maipu region of Mendoza. All of the clones were then subjected to the ELISA test to detect for Arabis mosaic virus, Grapevine fleck virus, Grapevine fanleaf virus, Grapevine leafroll associated virus Types 1, 2, 3, and Tomato ringspot virus. The clones were then multiplied from bi-nodal pruned budwood using micro-propagation techniques. This method ensured the overall good health of the multiplied plants.

A total of 51 to 55 plants from each clone successfully passed through the process of propagation and rustication, and having achieved the dimensions necessary to survive in the field, were planted in 1994 in Bodegas Esmeralda's La Piramide vineyard, located in the Agrelo district of the Lujan de Cuyo region of Mendoza. The Malbec grapevine clones were planted at a density of two meters between rows and 1.25 meters between plants and trained to a vertical shoot positioned trellis. They were pruned to a double Guyot system of loading canes with an average of 9 to 12 buds per cane.

The objective of the Malbec grapevine clonal selection was to identify those clones which possessed both vineyard performance and wine quality characteristics which were above the level achieved for the general population when working with massal selections of Malbec grapevines.

The vineyard performance criteria included overall grapevine development, shoot growth rate, plantation failures, dates of budbreak, veraison and harvest, compactness, size, number of clusters, size, number, and millendrage level of berries, susceptibility to mildew, as well as brix, pH, acidity and anthocyanin levels.

Given that the end goal of this selection process was to improve the overall quality of Malbec wine produced, certain elements of the above criteria were given more weight than others:

Color: Plants with overall high anthocyanin counts were given additional weight in the selection. Low anthocyanin count resulted in elimination from the selection.

Brix, Acidity and pH Levels: Proper development of these elements throughout the growing season as well as balance at the moment of harvest were important criteria for selection.

Berry Size: The selection process sought to identify Malbec grapevine clones with a high ratio of skin to juice, generally seeking smaller berries.

Millendrage: Malbec grapevine clones were chosen for low levels of millendrage and shot berries, seeking even fruit set.

In 1997 fifteen clones were selected with optimal vineyard performance (low yields, small clusters, small berries, high polyphenols and low millendrage) and varying yet complimentary flavor profiles. The fifteen clones were vinified separately and characteristics (aroma, concentration, natural acidity, ripening time, typicity, astringency, flavor sensation) were compared.

In 1998 the five best clones were selected using the joint vineyard and wine criteria described above. These clones were sent to University of Adelaide for virus testing, with all results negative.

In 1999 the original 108 clonal selection was planted in Bodegas Esmeralda's Adrianna Vineyard, located at 5,000 feet above sea level in the Gualtallary district of the Tupungato region in Mendoza, Argentina.

In 2002 using the same selection process as originally implemented in Bodegas Esmeralda's La Piramide vineyard, clones 13 and 17 were selected as optimum for this vineyard site and planted in an experimental 3 hectare block.

In 2003 the five selected clones were planted in Bodegas Esmeralda's Nicasia vineyard, located at 3,870 feet above sea level in the Altamira district of the San Carlos region in Mendoza.

In 2007 the five clones selected by Bodegas Esmeralda passed a three year viral field study conducted by the Foundation Plant Services Department at the University of California Davis. They have recently been released from quarantine after having passed all pertinent viral tests and are currently being held at Herrick Vines in California.

### DETAILED BOTANICAL DESCRIPTION

Below is a detailed botanical description of Catena Malbec ‘Clone 16’:
Vine generally:
   Size.—Medium. Grapevine size as determined on grapevines growing on a three wire vertical shoot positioned trellis with the first wire (fruit zone) set 80 cm (31.25 inches) above the ground; the second wire at 1.30 m (50.78 inches) above the ground; and the third wire at 1.8 m (70.31 inches) above the ground. The vine was trained to produce a grapevine height of 2.24 m (88.18 inches) and a grapevine spread of 36 cm (14.17 inches).
   Vigor.—medium vigor. Vigor as measured by weighing prunings at dormant pruning for cane pruned grapevines (with 13 canes and an average of 17 buds per cane) was 0.978 Kg.
   Productivity.—Productive. 1.4 Kg per grapevine as compared to the average population grapevine which produces 1.55 Kg per grapevine on grapevines spaced 4.1 ft. (125 cm) by 6.5 ft. (200 cm).
   Regularity of bearing.—Regular. Annual pruning of canes is required for reliable production.
Canes size:
   Diameter.—mature canes. — Medium diameter. medium vigor. upright in growth habit.
Mature canes:
   Diameter.—internode base. — 9 mm (0.351 inches).
   Diameter.—internode midpoint. — 8.2 mm (0.32 inches).
   Diameter.—internode tip. — 5.1 mm (0.199 inches).
   Diameter.—node base. — 11.4 mm (0.44 inches).
   Diameter.—node midpoint. — 13.6 mm (0.531 inches).
   Diameter.—node tip. — 7.1 mm (0.277 inches).
Internode length:
   Base.—6.9 cm (2.69 inches).
   Midpoint.—8.2 cm (3.20 inches).
   Tip.—8.35 cm (3.26 inches).
   Average length of canes.—138.4 cm (54.06 inches).
   Surface texture.—Smooth.
   Color of mature cane.—Brown. No anthocyanin observed on mature canes.
Buds:
   Color.—Brown.
   Texture.—Smooth.
Dormant bud (compound bud or eye):
   Width.—At base of cane 5 mm (0.195 inches); at midpoint of cane 5.3 mm (0.207 inches) and at tip of cane 3.8 mm (0.148 inches). The average number of buds on a current, single-season growth cane is 17.
   Date of bud break.—October 7 sup.rd. midseason.
   Young shoots.—Young shoots have cobwebby indument. Diameter of young shoots in spring (measured when shoots are 24 inches). — At base 7.1 mm (0.277 inches) at midpoint 5.4 mm (0.210 inches) and at tip 3.8 mm (0.148 inches).
   Internode length.—4.5 cm (1.75 inches) at 4.sup.th internode from base.
Young shoots:
   Color.—Pale yellow green.
Stem of shoot tip:
   Color.—Green with a slight copper tint in sun.
Shoot:
   Shape.—Straight to slightly curved.
Shoot tip:
   Form.—Open.
Tendrils:
   Size.—Length — 16.8 cm (6.56 inches).
   Size.—Diameter — 1.7 mm (0.066 inches).
   Shape.—Usually biforcated and curled on distal end.
   Pattern.—Found beginning opposite node 6 and 7, then again at nodes 9, 10, 12, 13, 15, 16 with this repeating intermittent pattern to the distal end of the cane.
Tendril:
   Color immature growth.—Yellow green with slight copper on tip.
   Disease resistance.—Susceptible to Odium and Mildew. and fungicides were applied to the grapevines under evaluation to control them.
   Insect resistance.—There has been no insect resistance detected given that insects are very rare in Mendoza.
LEAVES size:
   Generally.—Leaves simple and alternate. The mid vein (L1) is 13.5 cm (5.273 inches) long. vein L2 is 10 cm (3.906 inches) long and vein L3 is 7.2 cm (2.812 inches) long. The angle between the mid vein L1 and L3 is 61 degrees and between L1 and the 1st vein off L3 is 156 degrees.
   Average length.—17.2 cm (6.718 inches).
   Average width.—15 cm (5.86 inches).
   Shape.—Orbicular.
Lobes:
   Number.—five (5) three (3) without lobes.
Color:
   Upwardly disposed surface.—Dark green . Upward surface is glabrous. flat and smooth to slightly bullate.
   Downwardly disposed surface.—Lower surface has short hairs.
   Leaf vein.—Light green with occasional red on main veins near center of leaf.
   Leaf vein.—thickness. — Thickness of mid vein at center of leaf is 1.6 mm (0.062 inches).
   Leaf margin.—Serrated with shape of teeth pointed and medium in size (convex teeth).
   Petiole sinus.—Half open and “V” shape. On mature leaf is 4.4 cm (1.72 inches) deep and 1.3 cm (0.507 inches) wide at widest point.
Anthocyanin:
   Main veins.—location. — With occasional red on main veins near center of leaf.
Petiole:
   Size.—Medium.
   Length.—7.8 cm (3.04 inches).
   Diameter.—2.3 mm (0.089 inches).
   Color.—Green with occasional red covering.
Color:
   Young leaf.—upper surface. — Pale green with light copper and cobwebby indument on upper surface.
   Young leaf.—lower surface. — Shape unfolded — young leaf. — Concave to flat.
   Petiole of young leaf.—color. — Medium green.
   Stipules.—Onion skin.
Trunk size: Large.
   Height.—Approximately 75 cm (23.9 inches) above the vineyard floor.
   Diameter.—12.6 cm (4.92 inches) as measured just below the cordon or head point at 40 cm (15.6 inches) above vineyard floor.
Flowers Flower:
   Size.—generally. — Medium.
   Unopened.—diameter. — 2 mm (0.078 inches).
   Unopened.—length. — 1.85 mm (0.072 inches).
   Unopened.—surface texture. — Smooth.
   Date of bloom.—First bloom November 10.
   Date of full bloom.—November 17 at 90%.
   Inflorescence.—Panicle.
Cluster size.
   At bloom.—Generally. medium.
   Cluster.—length. — 13.1 cm (5.117 inches).
   Width.—11.5 cm (4.49 inches).
Peduncle:
   Length.—2.7 cm (1.054 inches).
   Shape of cluster.—Conical with shoulder well developed.
Calyptra:
   Color.—Green.
   Stamens.—Five (5) and erect.
   Pistil.—Well developed.
Ovary:
   Color.—Green.
   Pollen.—Normal. fertile. abundant.
Anthers:
   Color.—Straw.
Fruit: Maturity when descried: Ripe for commercial harvesting and shipment approximately March 10 in Mendoza. Argentina.
Cluster:
   Size.—cane pruned vines. — 119 grams (4.19 oz).
   Length.—14.63 cm (5.71 inches).
   Width.—13.4 cm (5.23 inches).
   Shape.—Conical.
   Density.—Tight. on average has 89 berries per cluster.
   Clusters per vine.—11.7.
   Clusters per shoot.—0.91 clusters per shoot.
Peduncle:
   Size.—Length. — Medium. 3.7 cm (1.44 inches).
   Diameter.—Medium. 5.1 mm (0.199 inches).
   Color.—Green.
   Texture.—Smooth. glabrous.
Pedicel:
   Generally.—There is a medium to good attachment between the berry and the pedicel.
   Size.—length. — 4.6 mm (0.179 inches).
   Size.—diameter. — 0.65 mm (0.025 inches).
   Color.—Green.
   Texture.—Glabrous.
Brush:
   Length.—2.15 mm (0.083 inches).
   Brush color.—Green.
Berry:
   Size.—Medium. avg. 0.99 grams (0.035 oz).
   Shape.—Spherical 1.15 cm (0.449 inches) long and 0.95 cm (0.371 inches) wide.
   Color.—Raspberry red Bloom. — Light.
Skin:
   Thickness.—Medium in thickness.
   Texture.—Smooth.
   Tendency to crack.—None.
Flesh:
   Flesh color.—Translucent and very pale yellow green.
   Texture.—Firm. meaty. Juice production. — High Color of juice. — Clear.
   Flavor.—Sweet. low acid flavor.
   Soluble solids.—25%.
   Titratable acid.—3.51 g/L ml juice.
   Aroma.—None.
   Ripening.—Uniform.
   Character of seeds.—Complete seeds. Seed color is auburn.
   Use.—wine.
   Resistance to disease.—No resistance to mildew and Odium.

Below are some comparative charts to demonstrate the differences found in the selected Malbec grapevine clones.

Table 1 shows how the Catena Malbec ‘Clone 16’ shows different physiological characteristics when compared to other Malbec grapevine clones selected by Bodegas Esmeralda as well as the overall massal population.

TABLE 1

| | Clone | Clone | Clone | Clone | Clone | Popula- |
|---|---|---|---|---|---|---|
| | 13 | 14 | 15 | 16 | 17 | tion |
| Potential | 4.2 | 6.2 | 3.6 | 4.1 | 5.3 | 3.62 |
| Foliage Surface | | | | | | |
| Area m2 | | | | | | |
| N° Shoots | 14.3 | 12.6 | 12.8 | 12.8 | 14.2 | 12.6 |
| Avg. Shoot | 123.0 | 152.1 | 76.0 | 138.4 | 141.4 | 94.3 |
| Length (cm) | | | | | | |
| Pruned | 1144.0 | 919.8 | 870.4 | 978.1 | 1202.3 | 917.4 |
| Material | | | | | | |
| Weight m/g | | | | | | |
| Exposed Sur- | 1.64 | 1.74 | 1.42 | 1.53 | 1.72 | 0.98 |
| face Area/ | | | | | | |
| Production | | | | | | |
| N° of Leaf | 3.6 | 2.5 | 2.6 | 2.9 | 3.3 | 2.7 |
| Layers | | | | | | |

### Clonal Individuality—Shotting (Millendrage)

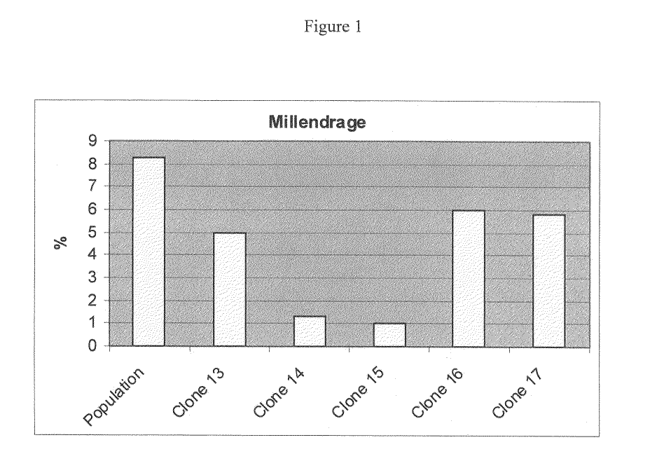

Malbec has a tendency for shot berries causing problems with homogeneity and cluster ripening. Homogeneity is a key factor for quality. See FIG. 1.

In addition, Table 2 shows some of the physiological characteristics and individuality of the Catena Malbec ‘Clone 16’, when compared to other selected Malbec grapevine clones and the overall Malbec grapevine massal population.

TABLE 2

| | Cluster | Cluster | | Total Berry | | No. of | Berry | | Compact |
|---|---|---|---|---|---|---|---|---|---|
| Clone | Length | Weight | | Weight | | Berries | Weight | | Index |
| 13 | 15.29 cm | 113.54 | gr | 110.11 | gr | 101.1 | 1.09 | gr | 0.15 |
| 14 | 16.84 cm | 128.9 | gr | 97.76 | gr | 97 | 1.01 | gr | 0.17 |
| 15 | 16.32 cm | 69.42 | gr | 69.75 | gr | 65.7 | 1.06 | gr | 0.25 |
| 16 | 14.63 cm | 119 | gr | 88 | gr | 89 | 0.99 | gr | 0.16 |
| 17 | 8.32 cm | 47.5 | gr | 37.8 | gr | 34.2 | 1.11 | gr | 0.24 |
| Population | 18.23 cm | 91.68 | gr | 85.06 | | 83.11 | 1.02 | | 0.22 |

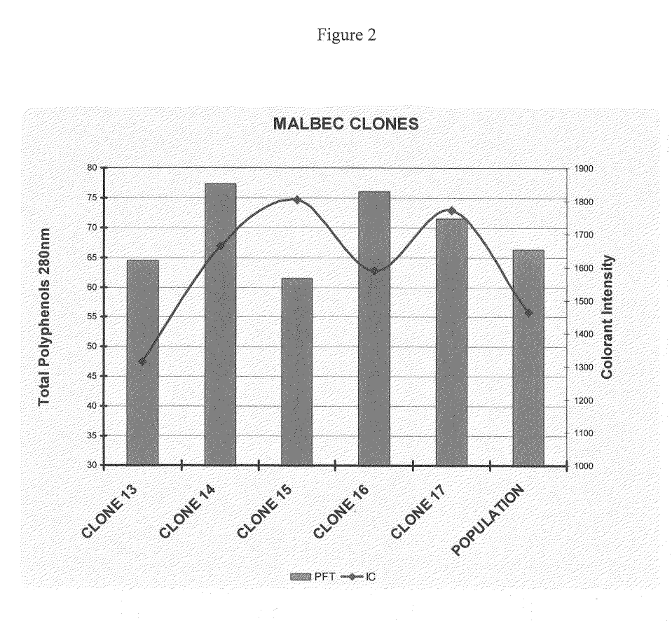

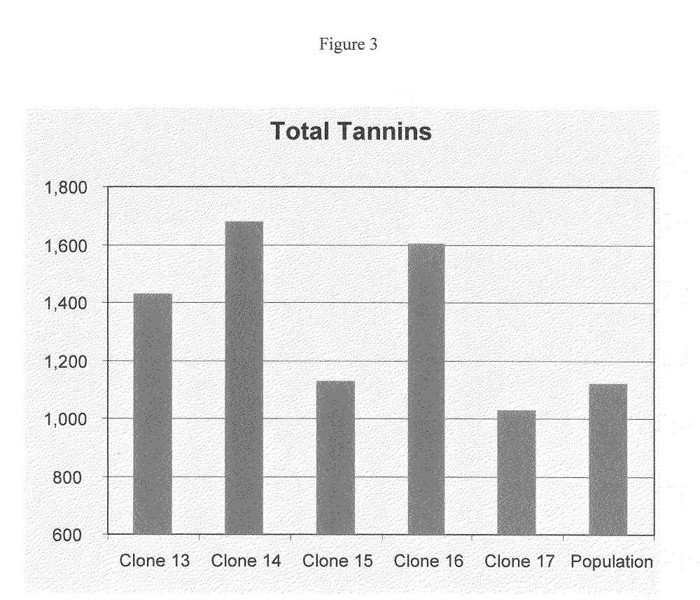

The individuality of this Malbec grapevine clone was also measured in terms of its chemical profile when compared to other selected Malbec grapevine clones and the overall Malbec grapevine massal population. Total polyphenols are shown in FIG. 2. Total tannins are shown in FIG. 3.

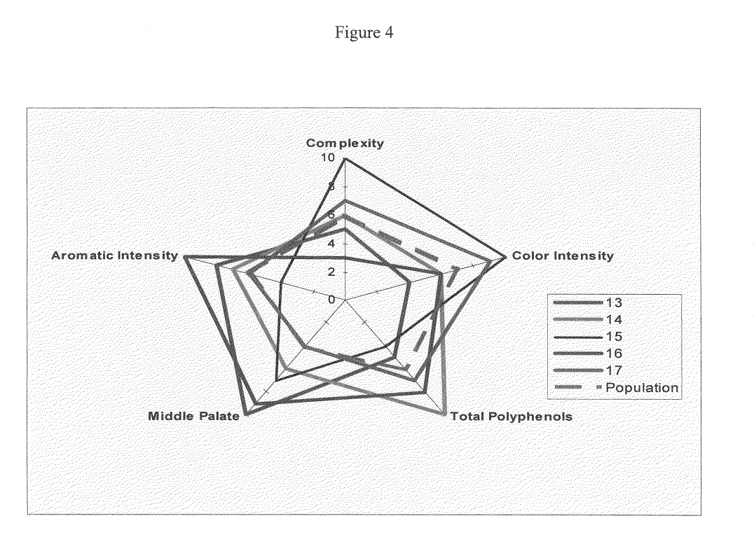

The Catena Malbec ‘Clone 16’ was also measured in terms of its aroma and flavor profile when compared to other selected Malbec grapevine clones and the overall Malbec grapevine massal population. The results are shown in FIG. 4.

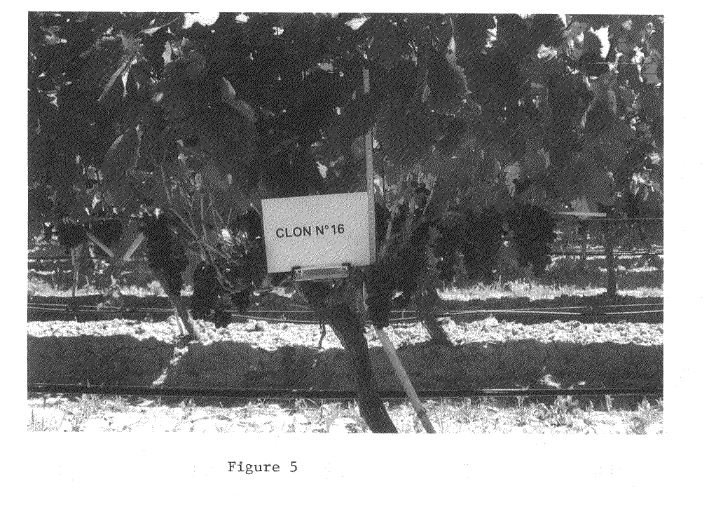

FIG. 5 is a photograph of the vine with fruit.

1. A novel and distinct variety of Malbec grapevine herein denominated ‘Catena Malbec Clone 16’ having the characteristics described and illustrated herein.
